(19) United States
(12) Patent Application Publication
(10) Pub. No.: US 2021/0045621 A1
(43) Pub. Date: Feb. 18, 2021

(54) CAPSULE ENDOSCOPE, RECEIVER INTERWORKING WITH CAPSULE ENDOSCOPE AND METHOD FOR CONTROL OF CAPSULE ENDOSCOPE

(71) Applicant: AJOU UNIVERSITY INDUSTRY-ACADEMIC COOPERATION FOUNDATION, Suwon-si (KR)

(72) Inventors: Junghyeon BAE, Suwon-si (KR); Myung Hoon Sunwoo, Seoul (KR)

(73) Assignee: AJOU UNIVERSITY INDUSTRY-ACADEMIC COOPERATION FOUNDATION, Suwon-si (KR)

(21) Appl. No.: 16/990,130

(22) Filed: Aug. 11, 2020

(30) Foreign Application Priority Data
Aug. 12, 2019 (KR) 10-2019-0098405

(51) Int. Cl. A61B 1/045; A61B 1/04; A61B 1/00; G06T 7/11; G06T 7/00

(52) U.S. Cl. CPC A61B 1/045; A61B 1/041; A61B 1/00045; A61B 1/00016; G06T 2207/30028; G06T 7/0012; G06T 2207/30096; G06T 2207/10068; G06T 7/11

(57) ABSTRACT

As a capsule endoscope control method in a receiving device interworking with the capsule endoscope, it is determined whether a lesion is detected in the digestive organ from the digestive organ image, upon receiving a digestive organ image for a digestive organ taken by the capsule endoscope. Depending on whether a lesion is detected and whether the digestive organ falls within a predetermined region of interest, a control signal is generated so that the capsule endoscope operates in one of a plurality of modes and transmitted to the capsule endoscope. Then, the capsule endoscope is controlled to capture the digestive organ in one of the plurality of modes.

### CROSS-REFERENCE TO RELATED APPLICATIONS

This application claims priority to and the benefit of Korean Patent Application No. 10-2019-0098405, filed in the Korean Intellectual Property Office on Aug. 12, 2019, the entire contents of which are incorporated herein by reference.

### BACKGROUND

### (a) Field

The present invention relates to a capsule endoscope, a receiving device interworking with the capsule endoscope, and a capsule endoscope control method.

### (b) Description of the Related Art

Despite asymmetry between transmission and reception of a signal in the digital/baseband, conventional medical capsule endoscopes use the same operating frequency while transmitting information obtained from the interior of a human body to an external system. Thus, redundant power consumption is provoked.

To solve such an issue, a capsule endoscope that adopts a human body communication (HBC) method employing a human body as a medium rather than an RF communication method has been developed. Due to requiring lower power than the RF communication method, the HBC communication method enables to acquire internal images of a human body for a longer time. However, in the HBC communication method transmitting high definition image information is impossible due to low bandwidth.

Additionally, a probability for a developed capsule endoscope to fail in detecting a lesion reaches up to 20 to 30%. Since the capsule endoscope moves only by the peristaltic motion of the gastrointestinal tract, a low power design is essential for activating a transceiver during many hours. Due to good peristaltic motion, an examination of a person in ages between teens and thirties takes around 10 hours. However, the examination of a person over 60 takes to a maximum of 24 hours. However, the battery capacity is limited due to the restriction on the size of the capsule endoscope. Thus, a probability of failure in detecting a lesion increases because of the limited operating time of the capsule endoscope.

Further, obtaining sufficient images of lesions is difficult in that the capsule endoscope obtains images at a fixed rate (for example, at a rate of 2-3 fps) and with a narrow viewing angle. In addition, since the capsule endoscope communicates passing through the human body, a probability of burst error increases.

### SUMMARY

Accordingly, the present invention provides a capsule endoscope supporting multi-mode image capture, a receiving device interworking with the capsule endoscope, and a capsule endoscope control method according to a patient's medical history and the presence of a lesion in real time.

According to an embodiment of the present invention, a capsule endoscope control method performed by a receiving device interworking with the capsule endoscope is provided. The receiving device may receive a digestive organ image of a digestive organ captured by the capsule endoscope, determine whether a lesion is detected in the digestive organ from the digestive organ image, generate a control signal so that the capsule endoscope operates in one of a plurality of modes, depending on whether a lesion is detected and whether the digestive organ falls within a predetermined region of interest, and transmits the control signal to the capsule endoscope and controlling the capsule endoscope to capture the digestive organ in one of the plurality of modes.

A first mode among the plurality of modes may be a mode that makes the digestive organ to be captured with a frame per second that is larger than a frame per second of a second mode. Further, the frame per second of the second mode may be smaller than the frame per second of the first mode, and the digestive organ may be captured with the frame per second that is larger than a frame per second of a third mode.

The frame per second of the first mode, the second mode and the third mode may be 10 fps, 5 fps, and 1 fps, respectively.

Generating the control signal may generating the control signal that makes the capsule endoscope operate in the first mode when it is determined that a lesion is detected from the digestive organ image.

Generating the control signal may include identifying whether the digestive organ is included in a predetermined region of interest, if it is determined that no lesion is detected from the digestive organ image, and generating the control signal that makes the capsule endoscope operate in the second mode when it is determined that the digestive organ is included in the predetermined region of interest.

The region of interest may include a small intestine, personal medical history information of a patient, and a digestive organ selected by the patient.

Generating the control signal comprises generating the control signal that makes the capsule endoscope operate in the third mode, when it is determined that the digestive organ is included in the region of interest.

According to another embodiment of the present invention, a receiving apparatus interworking with a capsule endoscope is provided. The receiving apparatus may include an interface that interworks with the capsule endoscope, receives an image signal from the capsule endoscope, and transmits to the capsule endoscope a control signal that is determined based on the image signal and controls a mode of the capsule endoscope, an image analysis unit that analyzes a digestive organ image captured by the endoscope, based on the image signal, and a mode determination unit that determines a mode in which the capsule endoscope operates, based on an analysis result of the digestive organ image analyzed by the image analysis unit and a region of interest input from the outside.

The mode determination unit may determine so that the capsule endoscope operates in a first mode, when a lesion is detected from the digestive organ image.

The mode determination unit may determine so that the capsule endoscope operates in a second mode, if no lesion is detected from the digestive organ image and the digestive organ is included in the region of interest.

The mode determination unit may determine so that the capsule endoscope operates in a third mode, if no lesion is detected from the digestive organ image and the digestive organ is not included in the region of interest.

According to a still another embodiment of the present invention, a capsule endoscope is provided. The capsule endoscope may include a first communication module that receives a control signal including mode information of the capsule endoscope from a receiving device interworking with the capsule endoscope, a second communication module that transmits a digestive organ image captured by the capsule endoscope to the receiving device, an image acquisition module that captures images of a digestive organ based on the mode information, a control module that controls the image acquisition module to capture images of the digestive organ based on the mode information received by the first communication module, and encrypts the digestive organ image captured by the image acquisition module, and a power supply module that supplies power to the first communication module, the second communication module, the image acquisition module, and the control module.

The first communication module may receive a control signal in a frequency band between 402 MHz and 405 MHz, and the second communication module may transmit to the receiving device the digestive organ image encrypted with the frequency band between 420 MHz and 450 MHz.

The image acquisition module may include a first image acquisition module that captures the digestive organ at a first position, and a second image acquisition module that captures the digestive organ at a second location different from the first location.

According to the present invention, since the capsule endoscope supports multi-mode image capture according to the presence of a lesion, unnecessary power consumption may be reduced.

### BRIEF DESCRIPTION OF THE DRAWINGS

FIG. 1 is an exemplary diagram of an environment where a capsule endoscope according to the present invention is utilized.

FIG. 2 is a structural diagram of a capsule endoscope according to an embodiment of the present invention.

FIG. 3 is a structural diagram of a control module according to an embodiment of the present invention.

FIG. 4 is an exemplary diagram illustrating a packet structure of a capsule endoscope according to an embodiment of the present invention.

FIG. 5 is a structural diagram of a receiving device according to an embodiment of the present invention.

FIG. 6 is a flowchart showing a control signal generating process in a receiving device according to an embodiment of the present invention.

### DETAILED DESCRIPTION OF THE EMBODIMENTS

Hereinafter, embodiments of the present invention will be described in detail with reference to the attached drawings so that the person of ordinary skill in the art may easily implement the present invention. The present invention may be modified in various ways and is not limited thereto. In the drawings, elements irrelevant to the description of the present invention are omitted for clarity of explanation, and like reference numerals designate like elements throughout the specification. Throughout the specification, when a part is referred to “include” a certain element, it means that it may further include other elements rather than exclude other elements, unless specifically indicates otherwise Hereinafter, a capsule endoscope and a power control method of the capsule endoscope according to embodiments of the present invention will be described in detail with reference to the accompanying drawings.

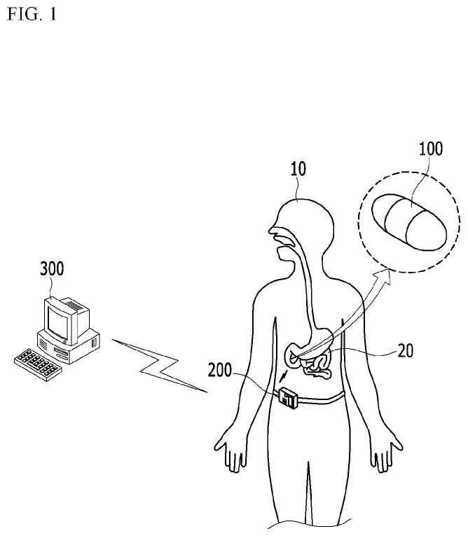

FIG. 1 is an exemplary diagram of an environment where a capsule endoscope according to the present invention is utilized.

As shown in FIG. 1, the environment includes a capsule endoscope 100 taken by a patient, a receiving device 200 that receives and transmits information obtained by the capsule endoscope 100 or relays a control signal to the capsule endoscope 100, and an information processing system 300 that processes the information obtained by the capsule endoscope 100.

The capsule endoscope 100 is in a form of a capsule, and implemented to be small-sized so as to be swallowed by a patient and to capture images of a digestive organ 20 in a human body 10. In the embodiment of the present invention, the size of the capsule endoscope 100 is not limited to a certain number.

When the patient swallows the capsule endoscope 100, a camera provided in the capsule endoscope 100 captures the interior of the digestive organ 20 while the capsule endoscope 100 moves from an esophagus to an anus through the gastrointestinal tract. Here, the type of the camera provided in the capsule endoscope 100 is not limited to a certain one.

The capsule endoscope 100 takes an internal image of the digestive organ 20 and then generates the image as an image signal. And also, the capsule endoscope 100 transmits the generated image signal to the receiving device 200 using a body conduction method that applies the human body 10 as a conductor. Here, the body conduction method is previously known to the person of ordinary skill in the art, so that a detailed description of the method is omitted.

The image signal transmitted by the capsule endoscope 100 is relayed to the receiving device 200 located outside the patient's body. The receiving device 200 reconstructs a digestive organ image from the image signal transmitted by the capsule endoscope 100. The receiving device 200 receives the image signal from the capsule endoscope 100 and stores the digestive organ image, until the capsule endoscope 100 completes image capture.

In addition, the receiving device 200 transmits to the capsule endoscope 100 a control signal so that the capsule endoscope 100 may operate in a multi-mode in the human body. In an embodiment of the present invention, a control signal that makes the capsule endoscope operate in one of the multi-mode including a fast mode, a normal mode, and a slow mode is transmitted. A detailed description thereof will be followed.

The receiving device 200 transmits the stored digestive organ image to the information processing system 300. In order to transmit and receive the digestive organ image, the receiving device 200 and the information processing system 300 are connected via a wired communication system or a short-range wireless communication system such as Bluetooth.

The information processing system 300 may regenerate the digestive organ image received from the receiving device 200 by frame-wise.

Further, the information processing system 300 may provide patient medical history information to the receiving device 200. In the embodiment of the present invention, the information processing system 300 may provide the patient medical history information to the receiving device 200, so that the capsule endoscope 100 may be controlled to take images of the digestive organ in the normal mode. Otherwise, a separate server (not shown) managing the medical history information may provide the medical history information to the receiving device 200.

In addition, when a user determines a portion of the patient's digestive organs as a region of interest, the information processing system 300 transmits information on the determined region of interest to the receiving device 200.

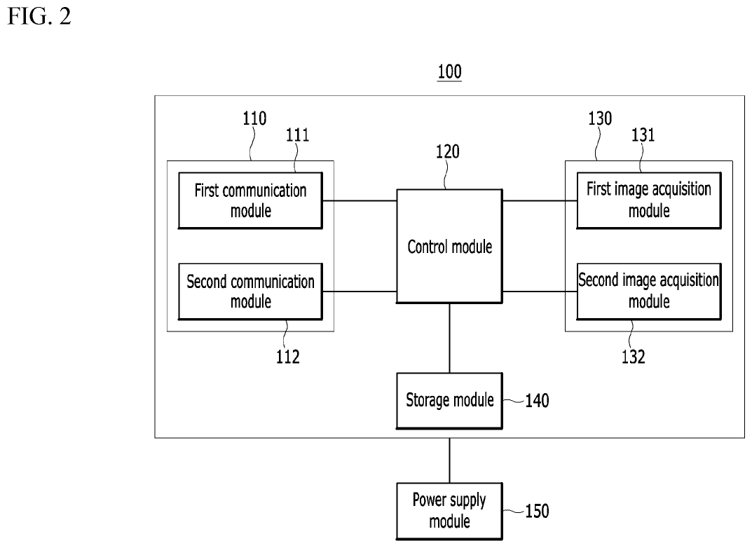

In this environment, a structure of the capsule endoscope 100 will be described with reference to FIG. 2.

FIG. 2 is a structural diagram of a capsule endoscope according to an embodiment of the present invention.

As shown in FIG. 2, the capsule endoscope 100 includes a communication module 110, a control module 120, and an image acquisition module 130.

The communication module 110 includes a first communication module 111 and a second communication module 112. The capsule endoscope 100 employs the first communication module 111 for receiving a signal transmitted from the receiving device 200 and the second communication module 112 for transmitting a signal to the receiving device 200.

The capsule endoscope 100 is a medical device licensed for using the medical device radio communication service (MICS) band (402-405 MHz). However, transmitting medical images with sufficient resolution is not guaranteed by using the licensed MICS band only. When a 10 bit Bayer filter type CMOS image sensor of QVGA (with a resolution of 320×240) class is used, the size of a raw medical image is 0.7 Mbit.

Since a viewing angle of a CMOS image sensor provided in a capsule endoscope ranges over only from 140 to 160 degrees, at least two image sensors are required. Further, when a lesion is detected, capturing images with a rate of 10 fps requires a bandwidth that enables to transmit images at a late of at least 7 Mb/s.

Therefore, in an embodiment of the present invention, the communication module 110 of the capsule endoscope 100 will be described as using an additional band besides the MICS band. In an embodiment of the present invention, a band of 420-450 MHz recommended by IEEE 802.15.6 WBAN will be described as an example of the additional band.

The capsule endoscope 100 is structurally asymmetric, in that a large amount of data is transmitted from inside to outside of a human body but a limited amount of data is transmitted in the opposite direction. Therefore, a first communication module 111 uses a first band (402-405 MHz) to receive a signal. In addition, a second communication module 112 in the capsule endoscope 100 uses, for example, 420-450 MHz to transmit image signal to the outside of the body, but it is not limited thereto.

The first communication module 111 receives a control signal from the receiving device 200. Here, the control signal includes an image capture request signal or a mode switch control signal for the capsule endoscope 100. The mode switch control signal is a signal that controls the capsule endoscope 100 to operate in a multi-mode in the human body according to the position of the capsule endoscope 100 or presence of a lesion.

In an embodiment of the present invention, the capsule endoscope 100 operates in one of a fast mode, a normal mode, and a slow mode according to a control signal.

When a lesion is detected, the capsule endoscope is determined to operate in the fast mode. Here, the fast mode is a mode for obtaining sufficient lesion images by capturing at a rate of 10 fps a digestive organ where the lesion is detected.

When the capsule endoscope 100 captures a small intestine, or a digestive organ having a medical history, or a region of interest such as a digestive organ of interest particularly input by a user, the capsule endoscope 100 is determined to operate in the normal mode. Here, the normal mode is a mode for obtaining images by capturing a digestive organ at a rate of 5 fps. Since for the conventional endoscope to access to the small intestine is difficult, diagnosing small intestine diseases is more difficult than other digestive organs. When capturing in the normal mode according to an embodiment of the present invention, it is possible to obtain internal images of the small intestine sufficiently.

The slow mode is a mode that enables to obtain images of remaining digestive organs not captured in the normal mode, such as stomach, esophagus and large intestine, and the like, by capturing at the rate of 1 fps.

The second communication module 112 uses the frequency of a second band, and transmits an image signal captured and encrypted by the capsule endoscope 100 to the receiving device 200.

When the first communication module 111 receives the control signal from the receiving device 200 via the first band, the control module 120 controls the image acquisition module 130 to capture an image of the digestive organ. The control module 120 may be divided into a receiving module for receiving a signal from the receiving device 200 and a transmitting module for transmitting a signal to the receiving device 200.

Upon receiving the control signal generated by the receiving device 200, the receiving module switches the mode of image capture in the capsule endoscope 100 and makes the image acquisition module 130 take images of the digestive organs of the patient. In addition, the transmitting module transmits an image signal generated by using the digestive organ image captured by the capsule endoscope 100 to the receiving device 200.

In an embodiment of the present invention, the image acquisition module 130 is described as to include a first image acquisition module 131 and a second image acquisition module 132, but it is not limited thereto.

The first image acquisition module 131 acquires internal images at a first position of the capsule endoscope 100, and the second image acquisition module 132 acquires digestive organ images at a second position of the capsule endoscope 100. The first image acquisition module 131 and the second image acquisition module 132 according to an embodiment of the present invention uses a CMOS image sensor and the viewing angle of one CMOS image sensor ranges over 140-160 degrees.

Therefore, it is possible to obtain images of the entire digestive organ through the two image acquisition modules 131 and 132. To this end, the first image acquisition module 131 and the second image acquisition module 132 are located so that the viewing angles thereof are not overlapped, and the locations thereof are not limited to certain points.

The storage module 140 stores the digestive organ images captured by the capsule endoscope 100 moving through the digestive organ until image capturing is completed. The stored image may be transmitted to the receiving device 200 at a predetermined period through the communication module 110. Otherwise, after the capsule endoscope 100 is discharged out of the body, the digestive organ images stored in the storage module 140 may be transferred to the data processing system 300 via a wired communication system.

The power supply module 150 supplies power to the capsule endoscope 100.

In the embodiment of the present invention, the type of the power supply module 150 is not limited to a certain one.

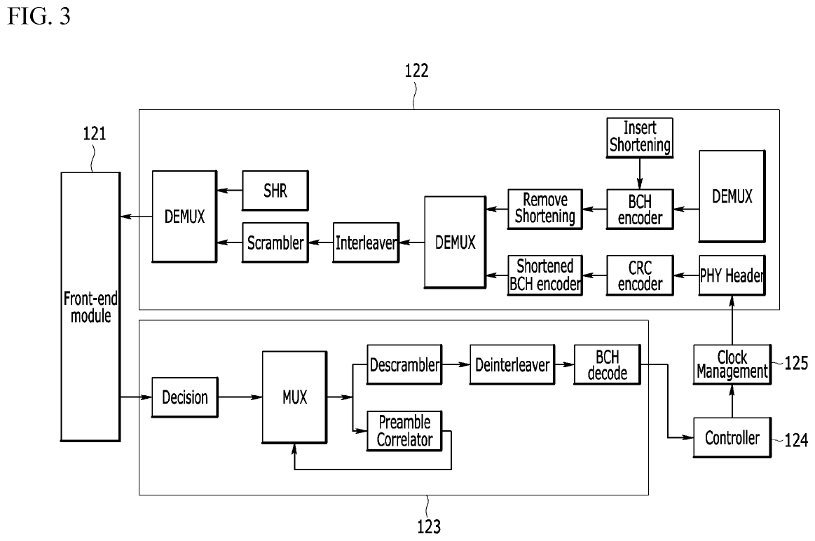

The control module 120 switches an image capture mode for the interior of a body based on the control signal generated by the receiving device 200 or processes the acquired digestive organ images. A structure of the control module 120 will be described in detail with reference to FIG. 3.

FIG. 3 is a structural diagram of a control module according to an embodiment of the present invention.

As shown in FIG. 3, the control module 120 may include a front-end module 121, a transmission signal processing module 122 that transmits a signal to a receiving device 200, a received signal processing module 123 that processes a received signal from the receiving device 200, a controller 124 and a clock management module 125.

Upon receiving a RF signal from the first communication module 111, the front-end module 121 generates I/Q signal through frequency shift keying (FSK) demodulation. The front-end module 121 may applies various methods to transform the RF signal to I/Q signal. Thus, a detailed description thereof will be omitted in an embodiment of the present invention.

Further, the front end module 121 performs FSK modulation on an encrypted digestive organ image that is to be transmitted from the capsule endoscope 100 to the receiving device, to generate and transmit an image signal in the form of a digital binary sequence. Generating the image signal through modulating the encrypted digestive image by the front-end module 121 may be implemented with various methods. Thus, a detailed description thereof will be omitted in an embodiment of the present invention. The transmission signal processing module 122 performs encryption of images obtained by the two image acquisition modules 131 and 132, respectively, through a first encryption module and a second encryption module. A Bose-Chaudhuri-Hocquenghem (BCH) encoder generates parity codes by encoding the encrypted digestive organ image.

Here, in order to minimize the power consumption in transmitting the image signal from the capsule endoscope 100, the BCH encoder receives a calculated shortening bit as an input before encoding the encrypted digestive organ image. For example, the shortening bit may be obtained through multiplying a codeword number per frame by BCH information bits and then subtracting bits per frame. Here, the codeword number per frame is obtained through dividing the number of bits used in one frame by information bits of the BCH code, but the methods for calculating the codeword number or shortening bits are not limited thereto.

When the BCH encoder generates parity codes by encoding the input shortening bits and the encrypted digestive organ images, the shortening bits that are not required for signal transmission are remained. Therefore, the parity codes output from the BCH encoder is transmitted to an interleaver after the shortening bits are removed.

Meanwhile, the transmission signal processing module 122 generates a PHY header of a 15-bit data unit to insert a header into the image signal. The methods for generating the PHY header or bit number of the PHY header are not limited to certain one.

Here, in order to protect the PHY header from errors, a cyclic redundancy check (CRC) encoder adds 4-bit of parity bits to the PHY header. In addition, in a shortening BCH encoder 12-bit of parity bits are further added to the header to which the parity bits are previously added.

The interleaver that receives the parity code from which the shortening bits are removed and the header added with parity bits performs channel interleaving on the received image signal. Then, a scrambler generates an image signal by randomly arranging bits. The generated image signal is transmitted by bit-wise in the order of header, encrypted frame body, and BCH parity to the transmitting device 200. Detailed description of the packet structure of the image signal will be followed.

The received signal processing module 123 receives the FSK demodulated I/Q signal in the front end module 121. A decision module detects a phase difference between I signal and Q signal and converts into a digital signal. A method for detecting the phase difference between I signal and Q signal in the decision module, and a method for converting I/Q signal into a digital signal are previously known, thus detailed description thereof will be omitted in the embodiments of the present invention.

A preamble correlator in the received signal processing module 123 checks whether a preamble of the converted digital signal is valid. In an embodiment of the present invention, a Kasami preamble recommended by IEEE 802.15.6 WBAN is described as an example of a valid preamble, but it is not limited thereto.

If the received signal processing module 123 determines that the preamble of the digital signal is valid, a descrambler removes the periodicity of the FSK demodulated signal. In addition, a deinterleaver and a BCH decoder correct a burst error that may occur when a signal passes through a human body.

The signal whose burst error is corrected is transmitted to a controller 124 as a control signal. The control signal is a signal indicating in which mode the capsule endoscope 100 operates.

The controller 124 controls the image acquisition module 130 to capture an internal image of the patient's body, based on the control signal received from the received signal processing module 123.

The clock management module 125 divides the system frequency (20 MHz) through the controller 124 based on packet information obtained by analyzing in the receiving device 200, and then transmits to the transmission signal processing module 122. At this time, in the embodiment of the present invention, the system frequency is divided into 1 MHz (slow mode), 5 MHz (normal mode), and 10 MHz (fast mode) and transmitted to the transmitting device, respectively.

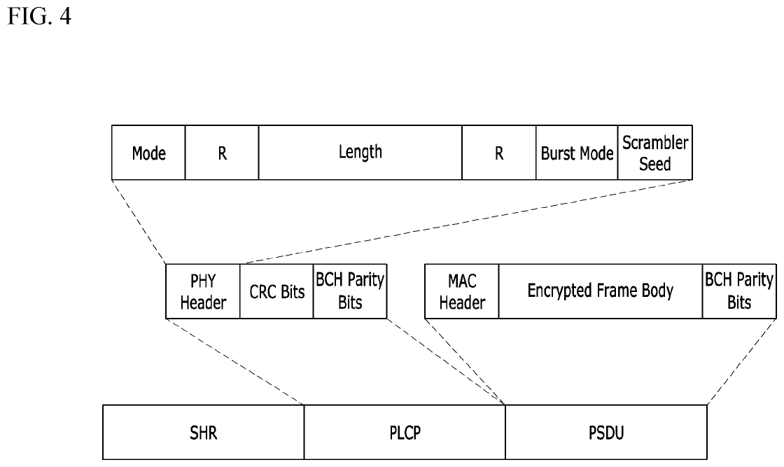

Hereinafter, a structure of the packet transmitted from the capsule endoscope 100 to the receiving device 200 will be described with reference to FIG. 4.

FIG. 4 is an exemplary diagram illustrating a packet structure of a capsule endoscope according to an embodiment of the present invention.

As shown in FIG. 4, the packet of the capsule endoscope 100 may include SHR, PLCP, and PSDU. The transmission signal processing module 121 transmits SHR, PLCP, and PSDU by one bit, respectively, according to a transmission order.

SHR includes a preamble and a start frame delimiter (SFD) so that the receiving device 200 can detect packets transmitted by the capsule endoscope 100. After SHR is transmitted to the receiving device 200, PLCP is transmitted.

In order to protect the PHY header of the PLCP from errors, the transmission signal processing module 121 insert 4 parity bits in the CRC encoder and then 12 parity bits in a shortened BCH encoder. The transmission signal processing module 121 adds 32 shortening bits to the connected PLCP, and then transmits by one bit to the receiving device 200. Here, all of the inserted shortening bits are “0”.

Firstly, the transmission signal processing module 121 transmits a MAC header indicating where the frame now being transmitted to the receiving device 200 is placed in the PSDU. Then, the transmission signal processing module 121 receives the captured images from the two image acquisition modules 131 and 132, encrypts the images using AES, and then transmits an encrypted frame body.

Thereafter, through a predetermined BCH encoding algorithm, 12 parity bits are added to the shortened BCH code and then transmitted to the receiving device 200 in parallel.

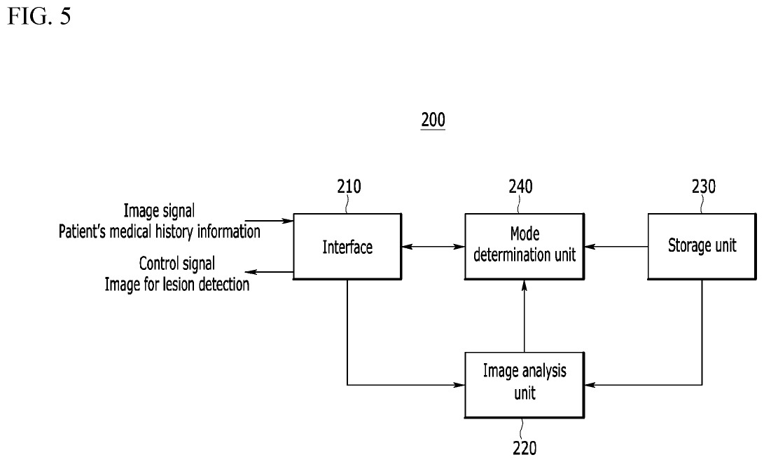

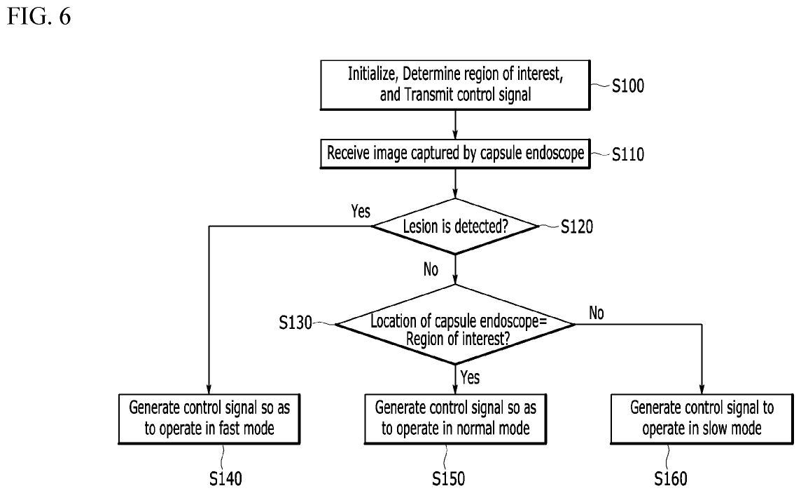

Next, a structure of a receiving device 200 determining in which mode a capsule endoscope 100 operates will be described with reference to FIG. 5, and a method for generating a control signal in a receiving device 200 will be described with reference to FIG. 6.

FIG. 5 is a structural diagram of a receiving device according to an embodiment of the present invention.

As shown in FIG. 5, the receiving device 200 includes an interface 210, an image analysis unit 220, a storage unit 230, and a mode determination unit 240.

In connection with the capsule endoscope 100, the interface 210 transmits a control signal to the capsule endoscope 100, or receives the image signal obtained by the capsule endoscope 100. In addition, the interface 210 also receives medical history information of a patient who swallowed the capsule endoscope 100 from the data processing device 300.

The image analysis unit 220 analyzes the image signal received through the interface 210. In addition, based on the analyzed image signal, the image analysis unit 220 determines whether a lesion exists in a digestive organ captured by the capsule endoscope 100 and which digestive organ the capsule endoscope 100 captured.

To this end, the image analysis unit 220 receives characteristic information of each digestive organ and patient medical history information stored in the storage unit 230, and determines the type of the digestive organ captured by the capsule endoscope 100. The characteristic information of each digestive organ, personal medical history information, and the method how the image analysis unit 220 determines the type of the captured digestive organ based on the characteristic information of each digestive organ are known in various ways. Thus, they are not limited to any one method or information type in the embodiment of the present invention.

Additionally, the image analysis unit 220 may provide a user with an analyzed image or an image signal received via the interface 210, through the data processing device 300. Further, if the image analysis unit 200 determines that a lesion exists, the analysis result is transmitted to the mode determination unit 240. Simultaneously, in order to verify the analyzed result, an image that is determined as having a lesion is transmitted to the data processing device 300.

The mode determination unit 240 determines in which mode the capsule endoscope 100 captures internal images of a body, based on personal medical history information of the patient stored in the storage unit 230, information of the presence of a lesion that is obtained through analyzing by the image analysis unit 22, or input from the outside.

That is, if it is analyzed that a lesion is present in the digestive organ captured by the capsule endoscope 100, the mode determination unit 240 determines so that the capsule endoscope 100 operates in the fast mode.

If the capsule endoscope 100 captured the small intestine or a digestive organ having medical history based on the personal medical history information stored in the storage unit 330, the mode determination unit 240 determines so that the capsule endoscope operates in the normal mode.

If it is determined that the capsule endoscope 100 captured digestive organs (for example, stomach, large intestine, esophagus, and the like) except for the digestive organs that are predetermined to be captured in the normal mode, the mode determination unit 240 determines so that the capsule endoscope 100 should operate in a slow mode.

The mode of the capsule endoscope 100 determined by the mode determination unit 240 is generated as a control signal and transmitted to the capsule endoscope 100 via the interface 210.

FIG. 6 is a flowchart showing a control signal generating process in a receiving device according to an embodiment of the present invention.

As shown in FIG. 6, the receiving device 200 transmits a control signal to be transmitted to the capsule endoscope 100, when the capsule endoscope 100 is initialized, or a user inputs a region of interest through the data processing system 300, or the region of interest is defined based on personal medical history information (S100).

In an embodiment of the present invention, a case where the slow mode is designated in an initially transmitted control signal is described as an example. However, if the region of interest is the esophagus, a control signal for the normal mode may be transmitted. In an embodiment of the present invention, the capsule endoscope 100 is instructed by the receiving device 200 to operate in three modes in the body considering the presence of a lesion and personal medical history information.

A first mode is a fast mode that is selected when a lesion is detected and makes the capsule endoscope 100 captures the interior of the body at the rate of 10 fps. A second mode is a normal mode that is selected when the capsule endoscope 100 passes through a small intestine or digestive organs having a medical history. In the normal mode, the capsule endoscope 100 captures the interior of the body at the rate of 5 fps. A third mode is a slow mode that is selected when the capsule endoscope 100 passes through other digestive organs (for example, stomach, esophagus, large intestine, and the like) not predetermined for the first and the second mode. In the slow mode the capsule endoscope 100 captures images at the rate of 1 fps.

When a lesion is detected, the capsule endoscope 100 captures lesion images at the rate of 10 fps to obtain sufficient lesion pictures using the fast mode. With the normal mode, the capsule endoscope 100 captures a digestive organ that has a past medical history but now does not have a detected lesion, or a spot that requires intensive capturing due to family history, at the rate of 5 fps. Finally, during most of the operation period the capsule endoscope 100 operates in the slow mode. Thus, the power consumption of the capsule endoscope 100 is minimized.

When the capsule endoscope 100 transmits the digestive organ image with a predetermined mode, the receiving device 200 receives the digestive organ image captured by the capsule endoscope 100 (S110). The receiving device 200 analyzes the received digestive organ image and determines whether a lesion is detected in the digestive organ photographed by the capsule endoscope 100 (S120).

If it is determined that a lesion is detected, the receiving device 200 generates and transmits a control signal so that the capsule endoscope 100 operates in the fast mode (S140). If it is determined that the lesion is not detected in step S120, the receiving device 200 checks whether the digestive organ captured by the capsule endoscope 100 is a digestive organ corresponding to a region of interest (S130).

In an embodiment of the present invention, the region of interest is a small intestine, a digestive organ having past medical history, or digestive organs that are selected as to be captured due to a family history by a user. When it is identified that the capsule endoscope 100 captured an image of the region of interest, the receiving device 200 generates a control signal so that the capsule endoscope 100 operates in the normal mode (S150). However, if it is determined that the digestive organ other than the region of interest is captured, the receiving device 200 generates a control signal to operate in the slow mode (S160).

As described above, by controlling the capsule endoscope 100 to operate in the multi-mode according to the medical history of a patient and the presence of lesions, unnecessary power consumption in capturing digestive organs can be reduced. In addition, since a clear image can be acquired for the region of interest, the accuracy of lesion detection may be increased.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A capsule endoscope control method performed by a receiving device interworking with the capsule endoscope, the method comprising:
receiving a digestive organ image of a digestive organ captured by the capsule endoscope;
determining whether a lesion is detected in the digestive organ from the digestive organ image;
generating a control signal so that the capsule endoscope operates in one of a plurality of modes, depending on whether a lesion is detected and whether the digestive organ falls within a predetermined region of interest; and
transmitting the control signal to the capsule endoscope and controlling the capsule endoscope to capture the digestive organ in one of the plurality of modes.

2. The method of claim 1, wherein a first mode among the plurality of modes is a mode that makes the digestive organ to be captured with a frame per second that is larger than a frame per second of a second mode,
the frame per second of the second mode is smaller than the frame per second of the first mode, and
the digestive organ is captured with the frame per second that is larger than a frame per second of a third mode.

3. The method of claim 2, wherein the frame per second of the first mode is 10 fps, the frame per second of the second mode is 10 fps, and the frame per second of the third mode 1 fps.

4. The method of claim 3, wherein generating the control signal comprises generating the control signal that makes the capsule endoscope operate in the first mode when it is determined that a lesion is detected from the digestive organ image.

5. The method of claim 4, wherein generating the control signal comprises:
identifying whether the digestive organ is included in a predetermined region of interest, if it is determined that no lesion is detected from the digestive organ image; and
generating the control signal that makes the capsule endoscope operate in the second mode when it is determined that the digestive organ is included in the predetermined region of interest.

6. The method of claim 5, wherein the region of interest includes a small intestine, personal medical history information of a patient, and a digestive organ selected by the patient.

7. The method of claim 5, wherein generating the control signal comprises generating the control signal that makes the capsule endoscope operate in the third mode when it is determined that the digestive organ is included in the region of interest.

8. A receiving apparatus interworking with a capsule endoscope, the apparatus comprising:
an interface that interworks with the capsule endoscope, receives an image signal from the capsule endoscope, and transmits to the capsule endoscope a control signal that is determined based on the image signal and controls a mode of the capsule endoscope;
an image analysis unit that analyzes a digestive organ image captured by the endoscope, based on the image signal; and
a mode determination unit that determines a mode in which the capsule endoscope operates, based on an analysis result of the digestive organ image analyzed by the image analysis unit and a region of interest input from the outside.

9. The apparatus of claim 8, wherein the mode determination unit determines so that the capsule endoscope operates in a first mode, when a lesion is detected from the digestive organ image.

10. The apparatus of claim 9, wherein the mode determination unit determines so that the capsule endoscope operates in a second mode, if no lesion is detected from the digestive organ image and the digestive organ is included in the region of interest.

11. The apparatus of claim 10, wherein the mode determination unit determines so that the capsule endoscope operates in a third mode, if no lesion is detected from the digestive organ image and the digestive organ is not included in the region of interest.

12. The apparatus of claim 11, wherein the region of interest includes a small intestine, personal medical history information of a patient, and a digestive organ selected by the patient.

13. A capsule endoscope comprising:
a first communication module that receives a control signal including mode information of the capsule endoscope from a receiving device interworking with the capsule endoscope;
a second communication module that transmits a digestive organ image captured by the capsule endoscope to the receiving device;
an image acquisition module that captures images of a digestive organ based on the mode information;
a control module that controls the image acquisition module to capture images of the digestive organ based on the mode information received by the first communication module, and encrypts the digestive organ image captured by the image acquisition module; and
a power supply module that supplies power to the first communication module, the second communication module, the image acquisition module, and the control module.

14. The capsule endoscope of claim 13, wherein the first communication module receives a control signal in a frequency band between 402 MHz and 405 MHz, and
the second communication module transmits to the receiving device the digestive organ image encrypted with the frequency band between 420 MHz and 450 MHz.

15. The capsule endoscope of claim 13, wherein the image acquisition module comprises
a first image acquisition module that captures the digestive organ at a first position, and
a second image acquisition module that captures the digestive organ at a second location different from the first location.
